(19) United States
(12) Patent Application Publication
(10) Pub. No.: US 2009/0142319 A1
(43) Pub. Date: Jun. 4, 2009

(54) METHOD TO EXPAND nTREG CELLS USING PI-3K ANTAGONIST

(75) Inventors: Steven Charles Eck, West Chester, PA (US); Li Li, Downingtown, PA (US)

Correspondence Address: HISCOCK & BARCLAY, LLP, 2000 HSBC PLAZA, 100 Chestnut Street, ROCHESTER, NY, 14604-2404, US

(73) Assignee: Therakos, Inc.

(21) Appl. No.: 12/364,659

(22) Filed: Feb. 3, 2009

Related U.S. Application Data
(63) Continuation-in-part of application No. 12/325,464, filed on Dec. 1, 2008.
(60) Provisional application No. 60/992,347, filed on Dec. 5, 2007.
(60) Provisional application No. 60/991,301, filed on Nov. 30, 2007.

(51) Int. Cl. A61K 35/12; C12N 5/06

(52) U.S. Cl. 424/93.71; 435/375

(57) ABSTRACT

Disclosed in this specification is a method to promote the growth of CD4+CD25Foxp3+ nTreg cells in a culture while treating with a PI-3K antagonist. The resulting cells are useful in the treatment of immune-related diseases.

### CROSS-REFERENCE TO RELATED APPLICATION

This application is a continuation-in-part of co-pending U.S. patent application Ser. No. 12/325,464 filed Dec. 1, 2008, which claims the benefit of U.S. provisional patent application Ser. No. 60/991,301, filed Nov. 30, 2007, and Ser. No. 60/992,347, filed Dec. 5, 2007, which applications are incorporated herein by reference in their entirety.

### FIELD OF THE INVENTION

This invention relates, in one embodiment, to a method for minimizing the growth of T effectors cells in cell populations during the expansion of nTreg cells. The resulting nTreg cells are particularly useful for treating immune diseases, such as graft versus host disease.

### BACKGROUND OF THE INVENTION

T regulatory (Treg) cells are important in maintaining the homeostatic balance of the human immune system and immune tolerance. One of the most well studied types of Treg cells is the natural Treg (nTreg) cell CD4+CD25+Foxp3+ cell. Defects in either the nTreg cells or in Foxp3 have been linked to unfavorable immune responses such as autoimmunity, allergic response, and organ rejection. Conversely, administration of healthy CD4+CD25+Foxp3+ nTreg cells have demonstrated therapeutic effects in the treatment of a variety of animal disease models. Although the nTreg cells are a small fraction of the circulating lymphocyte pool it has been found that nTreg cells can be expanded ex vivo to provide quantities of nTreg cells that are likely to be clinically useful. The possibility therefore exists for using ex vivo expanded nTreg cells to regulate the immune response of a human being.

During the process, nTreg cells are withdrawn from peripheral blood mononuclear cells (PBMC) using magnetic bead-based methods. The enriched nTreg cells are activated with anti-CD3/CD28 coated beads in the presence of high concentrations (ca. 1000 U/ml) of human recombinant IL-2. Although the purified cells are enriched for nTreg using the bead-base methods, the resulting sample is not pure. Due to the lack of nTreg-specific surface markers, the sample almost always contains non-Treg cells that expressed similar cell surface markers (e.g. CD4 and CD25). Indeed impurities are almost always present regardless of the purification method used. After about three weeks of culture time, the nTreg cell populations underwent multiple folds of expansion while maintaining their phenotypic expressions (CD25+ Foxp3+). Careful culturing conditions are needed to prevent decreases in the percent of cells expressing the Foxp3 protein. The most likely explanation for the decrease in the percent of cells expressing the Foxp3 protein is more rapid expansion of non-nTreg cells than the nTreg cells disturbing the overall composition of the sample. It is conceivable that loss of Foxp3 expressing cells in the composition could also be in part a result of some initially Foxp3 expressing cells losing expression of Foxp3 during the culture process. The overgrowth of non-Treg cells during Treg expansion not only potentially reduces the potency and effectiveness of the Treg cell therapy, but also provides a potential source of pro-inflammatory T effector cells and cytokines. Thus there is a need to find strategies and compounds to suppress the activation and growth of non-Treg cells and/or promote preferential maintenance and/or expansion of Treg cells in the cultured population.

### SUMMARY OF THE INVENTION

Applicants have discovered that inhibition of the PI-3K pathway allows for the preferential growth of nTreg cells over T effectors cells during expansion of the population. The resulting cells are useful for treating a variety of immune-related diseases, such a graph-versus-host disease. Additionally, applicant has discovered that the beneficial effectors of certain immune suppression agents (such as rapamycin and Ly294002) are synergistically or additively improved when the agents are used in conjunction with certain steroids (such as dexamethasone and prednisolone).

Disclosed in this specification is a method for expanding a population nTreg cells preferentially over T effectors cells by using an inhibitor of PI-3K.

### BRIEF DESCRIPTION OF THE DRAWINGS

The present invention is disclosed with reference to the accompanying drawing, wherein:

FIG. 1 is a graphical depiction of the response of nTreg cells to PI103.

Corresponding reference characters indicate corresponding parts throughout the several views. The examples set out herein illustrate several embodiments of the invention but should not be construed as limiting the scope of the invention in any manner.

### DETAILED DESCRIPTION

Human natural Treg (nTreg) cells were purified from normal donor PBMC using the commercially available Miltenyi Treg kit with AutoMacs (available from Miltenyi). The resulting sample was enriched in nTreg cells relative to the original sample. Similar results were obtained by depleting CD19+ cells and thereafter performing positive selection of CD25+ by AutoMacs using Miltenyi CD19 and CD25 beads. Typically, 50-70% of the enriched cells are Foxp3+ as assessed by inteacellular Foxp3 staining and flow cytometry analysis. In some studied, nTreg was enriched through FACS sorting based on CD4+, CD25high and CD127lo population. In this case, the Foxp3+ cells consisted of greater than 90% of the CD4+CD25+ population. After expansion for three weeks time, nTreg cells experience over a one hundred fold population growth. About 50% of the expanded cells were Foxp3+ and the cultured cells exhibited potent inhibitory activities during in vitro functional assays.

It has been reported (Bensinger et al. “Distinct IL-2 receptor signaling pattern in CD4+CD25+ regulator T cells” J. Immunol 172(9): 5287-96 (2004)) that Treg cells exhibit reduced AKT activities compared with the activities of the convention effector cells under certain conditions. It was believed that the proposed signaling pathway was PI-3K→AKT→mTOR. Without wishing to be bound to any particular theory, applicants believe that T effector cells are sensitive to upstream/downstream inhibitors of the PI-3K pathway. Accordingly, inhibition of this pathway during the expansion of the population of nTreg cells may allow for the preferential growth of nTreg cells. Several inhibitors of the PI-3K-AKT pathway were tested for activity during nTreg cell expansion, including LY294002.

LY294002 (2-(4-Morpholinyl)-8-phenyl-4H-1-benzopyran-4-one) is a cell-permeable, potent, and specific phosphatidylinositol 3-kinase inhibitor (IC₅₀=1.4 μM) that acts on the ATP-binding site of the enzyme. It also inhibits nonhomologous DNA end-joining (NHEJ) activity of the 460 kDa phosphatidylinositol 3-like kinase DNA-PKcₛ, which is the catalytic subunit of DNA-activated protein kinase. It does not affect the activities of EGF receptor kinase, MAP kinase, PKC, P1 4-kinase, S6 kinase, and c-Src even at 50 μM. It also blocks the proliferation of cultured rabbit aortic smooth muscle cells without inducing apoptosis (CAS 154447-36-6).

Several concentrations of LY294002 (2-(4-Morpholinyl)-8-phenyl-4H-1-benzopyran-4-one, 10 mM in DMSO, Calbiochem product #440204) ranging from 50 μM to roughly 100 nM final concentration) was added to select cultures. Concentrations greater than 50 μM were generally overly toxic to the cells. Control cultures included no additives and 100 nM rapamycin. Cultures were stimulated with anti-CD3-CD28 coated beads (Dyna1) at 3:1 bead to cell ratio in the presence of 100 IU/ml of human r-IL-2 and placed in a 37° C. incubator. Fresh IL-2 was supplied at the end of the first week and subsequently changed every three days or as needed. The purity of the nTreg cells was checked on days 6-7 of the culture and weekly thereafter.

TABLE 1

| | |
|---|---|
| Additive | % Foxp3+ |

Example Experiment A

| | |
|---|---|
| None | 16% |
| Ly294002 (10 μM) | 45 |
| Rapamycin (100 nM) | 45 |

Example Experiment B

| | |
|---|---|
| LY 294002 (10 μM) | 51% |
| Prednisolone (400 ng/ml) | 37% |
| Ly294002 (10 μM) + Prednisolone 3 ng/ml | 52% |
| Ly294002 (10 μM) + Prednisolone 50 ng/ml | 59% |
| Ly294002 (10 μM) + Prednisolone | 61% |
| 200 ng/ml | |
| Ly294002 (10 μM) + Prednisolone | 61% |
| 400 ng/ml | |

Example Experiment C:

| | |
|---|---|
| No additive | 56% |
| LY294002 (10 μM) | 82% |
| Dexamethasone (100 μM) | 63% |
| Ly294002 (10 μM) + Dexamethasone | 89% |
| (100 μM) | |

Example Experiment D:

| | |
|---|---|
| No additive | 34% |
| Rapamycin (100 nM) | 56% |
| Dexamethasone (100 μM) | 22% |
| Rapamycin (100 nM) + Dexamethasone | 82% |
| (100 μM) | |

Example Experiment E:

| | |
|---|---|
| Rapamycin (100 nM) | 63% |
| Rapamycin (100 nM) + Dexamethasone | 77% |
| (100 μM) | |
| Rapamycin (100 nM) + Prednisolone (200 ng/ml) | 83% |

Example Experiment F:

| | |
|---|---|
| No additive | 36% |
| AS252424 (1.25 ng/ml) | 60.1% |
| AS605240 (400 ng/ml) | ?58% |

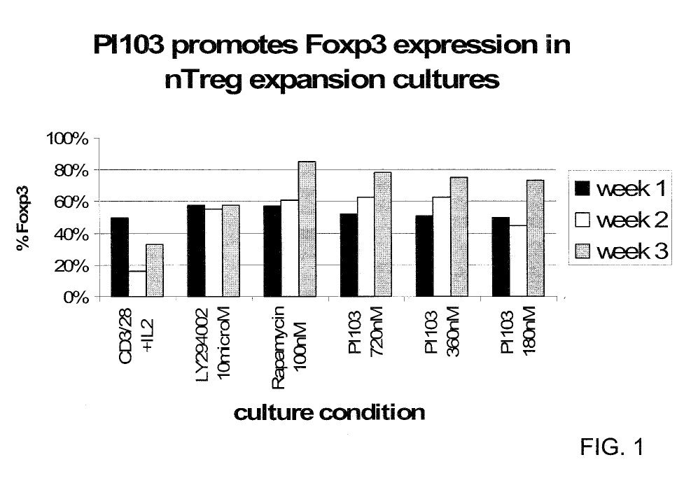

Other PI-3K inhibitors were also tested include PI-103. PI-103, (P3-(4-(4-Morpholinyl)pyrido[3,2:4,5]furo[3,2-d]pyrimidin-2-yl)phenol) is a cell-permeable pyridinylfuranopyrimidine compound that acts as a potent and ATP-competitive inhibitor of DNA-PK, PI3-K, and mTOR (IC₅₀=2, 8, 88, 48, 150, 26, 20, and 83 nM for DNA-PK, p110α, p110β, p110δ, p110γ, PI3-KC2b, mTORC1, and mTORC2, respectively). It inhibits ATR and ATM only at much higher concentrations (IC₅₀=850 and 920 nM, respectively) and exhibits little activity towards a panel of more than 40 other kinases even at concentrations as high as 10 μM. It has also been shown to effectively block PI3-K/Akt signaling and cell proliferation in glioma cell lines both in vitro and in vivo (CAS 371935-74-9). As shown, addition of PI103 at 720 nM, 360 nM or 180 nM were able to enhance Foxp3 expression as compared to their absence over a 3 week period of expansion culture of nTreg demonstrating the potential usefulness of this compound in the in vitro expansion of regulatory T cells as defined by a preculture presence of Foxp3 expression. See FIG. 1.

Wortmannin as a broad and irreversible PI3K inhibitor was also tested and found to promote a dose dependent increase in Foxp3 expression between 12.5 and 50 μM. AS 605240 is a potent, cell permeable and ATP-competitive inhibitor of phosphoinositide 3-kinase □(PI(3)K□). It exhibits selectivity over other PI(3)K isoforms. AS 252424 is a potent and selective phosphoinositide 3-kinase (PI(3)K) p110□ inhibitor shown to be selective for class IB PI(3)K-mediated cellular effects. are also PI3K inhibitors and were able to increase the percentage of cells in culture expressing Foxp3 relative to the respective compounds absence.

Interestingly, LY303511 (a derivative of LY294002) has been described to have an inhibitory effect on mTOR similar to that described for LY294002 but does not inhibit PI-3K. LY303551 did not permit for the selective growth of nTreg cells like its counterpart LY294002. AKT has been widely described as linking PI3K signal transduction to mTOR complexes. In this respect it is interesting that the AKT inhibitors that were tested were largely ineffective at promoting the expansion of nTreg cells. Tested described AKT inhibitors include but are not limited to Akt Inhibitor VII, TAT-AKT-in (Calbiochem product #124014); AKT inhibitor VIII, Isozyme-Selective, AKTi-½ Calbiochem product #124018; and AKT inhibitor X (10(4-(N-diethylamino)butyl)-2-chlorophenoxazine, HCl; Calbiochem product #124020).

Additionally, the use of steroids (e.g. dexamethasone or prednisolone) had a synergistic or additive effect with respect to the % of Foxp3 expressing cells obtained in expansion cultures when used with either rapamycin or LY294002. This effect was optimal around 200 ng/ml (prednisolone) or 100 μM (dexamethasone).

One of the characteristics of CD25+ and/or Foxp3+ CD4 T cells is their ability to suppress CD4 T cell responses to antigen-specific as well as anti-CD3 stimulation in the presence of antigen presenting cells. Similarly, CD25+ cells expanded in vitro have also been described as having similar suppressive capabilities. Inclusion of rapamycin is such expanded cultures, in addition to enhancing Foxp3 expression, also has been shown to enhance the suppressive capability of the expanded cells. Applicants have discovered that LY294002 has a similar effect when combined with the expanded CD25+ cells of the invention.

Treg cultures were established as described elsewhere in this specification and expanded for two weeks. Cells were harvested, washed two times and serial diluted onto a 96-well round-bottom plate. CFSE labeled purified CD4T cells were added to the wells at 1×10⁵ cells per well to result in the Treg:CD4 responder cell ratio's shown in Table 2. Cultured Dendritic cells were added at 1×10⁴ cells per well and OKT3 mAb was added at 1 μg per ml. Percent response indicates the percentage of CFSE labeled CD4T responder cells that failed to divide in the assay.

TABLE 2

| No additive | LY294002 (10 μg per ml) | Treg:CD4resp |
|---|---|---|
| (% response) | (% response) | ratio |
| 75% | 88% | 1:2 |
| 52% | 85% | 1:4 |
| 51% | 75% | 1:8 |
| 31% | 56% | 1:16 |

| | |
|---|---|
| 31% (CD4resp + OKT3 + DC) | 0:1 |
| 97% (CD4resp + OKT3, no DC) | NA |

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments failing within the scope and spirit of the appended claims.

What is claimed is:

1. A process for producing CD4+CD25+ nTreg cells comprising the steps of:
enriching CD4+CD25+ regulatory T cells in a sample thus producing enriched CD4+CD25+ regulatory T cells;
expanding the population of the enriched CD4+CD25+ regulatory T cells while treating the enriched cells with a PI-3K inhibitor; and
administering a portion of the expanded CD4+CD25+ regulatory T cells to a human being to treat graft versus host disease.

2. The process as recited in claim 1, wherein the step of expanding the population is performed for at least one week, but less than three weeks.

3. The process as recited in claim 1, wherein the step of expanding the population is performed for at least five days, but less than four weeks.

4. The process as recited in claim 1, wherein the step of expanding the population is performed for about two weeks.

5. The process as recited in claim 1, wherein the step of enriching the CD4+CD25+ regulatory T cells produces an enriched sample that is 35% to 90% CD4+CD25+ regulatory T cells relative to the total cell population in the enriched sample.

6. The process as recited in claim 1, wherein, after the step of expanding the population, the sample is 40% to 78% CD4+CD25+ regulatory T cells relative to the total cell population.

7. The process as recited in claim 1, wherein the concentration of the CD4+CD25+ regulatory T cells in the sample, both before and after expansion, are equal within a range of about 10%.

8. The process as recited in claim 1, wherein the step of expanding the population is performed for a sufficient period of time to result in a fold change in cell population ranging from not less than 30 fold increase to not greater than 300 fold increase.

9. The process as recited in claim 1, further comprising the step of administering a portion of the expanded CD4+CD25+ regulatory T cells to a human being to treat graft versus host disease.

10. A process for producing CD4+CD25+ nTreg cells comprising the steps of:
enriching CD4+CD25+ regulatory T cells in a sample thus producing enriched CD4+CD25+ regulatory T cells;
expanding the population of the enriched CD4+CD25+ regulatory T cells while treating the enriched cells with a steroid selected from the group consisting of dexamethasone and prednosolone and with an immune suppressive agent selected from the group consisting of rapamycin and Ly294002.

11. The process as recited in claim 10, wherein the concentration of the CD4+CD25+ regulatory T cells in the sample, both before and after expansion, are equal within a range of about 10%.
